(19) United States
(12) Patent Application Publication
(10) Pub. No.: US 2023/0108134 A1
(43) Pub. Date: Apr. 6, 2023

(54) DETERIORATION DIAGNOSIS DEVICE, AND RECORDING MEDIUM

(71) Applicant: NEC Corporation, Minato-ku, Tokyo (JP)

(72) Inventors: Nana JUMONJI, Tokyo (JP); Chisato SUGAWARA, Tokyo (JP)

(73) Assignee: NEC Corporation, Minato-ku, Tokyo (JP)

(21) Appl. No.: 17/908,653

(22) PCT Filed: Mar. 8, 2021
(86) PCT No.: PCT/JP2021/009038

§ 371 Date: Sep. 1, 2022

(30) Foreign Application Priority Data
Mar. 31, 2020 (JP) 2020-062913

(51) Int. Cl. G06T 7/00

(52) U.S. Cl. CPC G06T 7/0002

(57) ABSTRACT

A deterioration diagnosis device according to the present invention includes: a memory; and at least one processor coupled to the memory. The processor performs operations. The operations include: storing deterioration degree histories for portions of a structure to be diagnosed; acquiring reference information related to deterioration of the portions; generating a deterioration prediction model for predicting the deterioration degrees of the portions based on the histories and the reference information; predicting the deterioration degrees of the portions at a prediction time by using the generated deterioration prediction model; selecting, from among the portions, a portion where the deterioration degree predicted at the prediction time meets a predetermined condition; and outputting information relating to the selected portion and the predicted deterioration degree of the selected portion.

### TECHNICAL FIELD

The present invention relates to diagnosis of deterioration of a structure such as a road surface.

### BACKGROUND ART

Structures such as a road surface of a road, a sign installed on a road side, and a ceiling and a side wall of a tunnel or the like deteriorate over time.

Therefore, a device that predicts deterioration of a structure or the like has been proposed (see, for example, PTLs 1 and 2).

An image processing apparatus described in PTL 1 synthesizes damage with respect to images captured by division, and corrects a deterioration prediction model based on the synthesized damage.

A road management system described in PTL 2 predicts the road surface state based on changes in road surface state and transitions of traffic volume during a measurement period.

### Citation List

### Patent Literature

[PTL 1] WO 2019/163329 A [PTL 2] JP 2019-185443 A

### SUMMARY OF INVENTION

### Technical Problem

By using the techniques described in PTLs 1 and 2, a user can predict a deterioration state at a predetermined time.

However, such a generally used deterioration diagnosis device for a road surface diagnoses deterioration for each predetermined portion (e.g., 100 m) as a deterioration diagnosis of a road surface or the like using an image.

On the other hand, there are several hundred kilometers to several thousand kilometers of roads to be managed. Therefore, the number of portions to be subjected to the deterioration diagnosis is considerable.

Many steps have been required for a repair manager to select a portion to be preferentially repaired (e.g., the most deteriorated portion at a prediction time) among from the deterioration predictions of a plurality of portions at the prediction time output from the deterioration diagnosis device or the like.

The techniques described in PTLs 1 and 2 can predict deterioration, but have an issue in that a portion to be preferentially repaired cannot be selected from among the predicted portions.

Therefore, it is desired to select a portion meeting a predetermined condition at a prediction time from among portions for which deterioration has been predicted.

An object of the present invention is to solve the above-mentioned issue and to provide a deterioration diagnosis device or the like that selects a portion meeting a predetermined condition at a prediction time from among portions for which deterioration has been predicted.

### Solution to Problem

A deterioration diagnosis device according to an example aspect of the present invention includes:
a deterioration information storage means configured to store deterioration degree histories for portions of a structure to be diagnosed;
a reference information acquisition means configured to acquire reference information related to deterioration of the portions;
a model generation means configured to generate a deterioration prediction model for predicting the deterioration degrees of the portions based on the histories and the reference information;
a deterioration prediction means configured to predict the deterioration degrees of the portions at a prediction time by using the generated deterioration prediction model;
a portion selection means configured to select, from among the portions, a portion where the deterioration degree predicted at the prediction time meets a predetermined condition; and
an output means configured to output information relating to the selected portion and the predicted deterioration degree of the selected portion.

A deterioration diagnosis system according to an example aspect of the present invention includes:
the deterioration diagnosis device described above;
an information providing device configured to provide reference information to the deterioration diagnosis device;
an input device configured to transmit a prediction time to the deterioration diagnosis device; and
a display device configured to display the deterioration degree at a prediction time of the output portion by using information related to the portion and the deterioration degree output from the deterioration diagnosis device,.

A deterioration diagnosis method according to an example aspect of the present invention includes:
storing deterioration degree histories for portions of a structure to be diagnosed;
acquiring reference information related to deterioration of the portions;
generating, based on the histories, a deterioration prediction model for predicting the deterioration degrees of the portions;
predicting the deterioration degrees of the portions at a prediction time by using the generated deterioration prediction model;
selecting, from among the portions, the portion where the deterioration degree predicted at the prediction time meets a predetermined condition; and
outputting information relating to the selected portion and the predicted deterioration degree of the selected portion.

A recording medium according to an example aspect of the present invention stores a program for causing a computer to execute:
a process of storing deterioration degree histories for portions of a structure to be diagnosed;
a process of acquiring reference information related to deterioration of the portions;
a process of generating a deterioration prediction model for predicting the deterioration degrees of the portions based on the histories and the reference information;
a process of predicting the deterioration degrees of the portions at a prediction time by using the generated deterioration prediction model;
a process of selecting, from among the portions, the portion where the deterioration degree predicted at the prediction time meets a predetermined condition; and
a process of outputting information relating to the selected portion and the predicted deterioration degree of the selected portion.

### Advantageous Effects of Invention

According to the present invention, the effect of selecting, from among portions for which deterioration has been predicted, a portion meeting a predetermined condition at a prediction time can be exerted.

### BRIEF DESCRIPTION OF DRAWINGS

FIG. 1 is a block diagram illustrating an example of a configuration of a deterioration diagnosis system including a deterioration diagnosis device according to a first example embodiment.

FIG. 2 is a flowchart illustrating an example of operation of the deterioration diagnosis device.

FIG. 3 is a diagram illustrating an example of display for inputting a prediction time.

FIG. 4 is a diagram illustrating another example of display for inputting a prediction time.

FIG. 5 is a diagram illustrating a first example of display of portions.

FIG. 6 is a diagram illustrating a second example of display of the portions.

FIG. 7 is a diagram illustrating a display example of predetermined deterioration.

FIG. 8 is a diagram illustrating examples of displaying selected portions.

FIG. 9 is a block diagram illustrating an example of a hardware configuration of the deterioration diagnosis device.

FIG. 10 is a block diagram illustrating an example of a configuration of a deterioration diagnosis device according to a second example embodiment.

FIG. 11 is a flowchart illustrating an example of operation of the deterioration diagnosis device according to the second example embodiment.

FIG. 12 is a block diagram illustrating an example of a configuration of a deterioration diagnosis system including the deterioration diagnosis device according to the second example embodiment.

FIG. 13 is a diagram illustrating an outline of Intelligent Transport System (ITS).

### EXAMPLE EMBODIMENTS

Next, example embodiments of the present invention will be described with reference to the drawings.

Each drawing is for describing an example embodiment of the present invention. However, the present invention is not limited to the description of each drawing. In addition, similar configurations in the drawings are denoted by the same reference numerals, and repeated description thereof may be omitted. In addition, in the drawings used in the following description, the description of parts not related to the description of the present invention may be omitted and not illustrated.

### Terms

First, terms in the description of each example embodiment will be described.

The “deterioration degree” is a result of deterioration diagnosis (e.g., the extent of deterioration) for portions of a structure to be diagnosed.

The way of expressing “deterioration degree” is optional. For example, a numerical value may be used for expressing the deterioration degree. Alternatively, a value other than a numerical value may be used for expressing the deterioration degree. For example, characters such as {SMALL, MEDIUM, LARGE} may be used for expressing the deterioration degree.

In each example embodiment, a predetermined analysis method is applied to an image including portion of structure to be diagnosed to calculate the deterioration degree of each portion. A target structure of each example embodiment is optional. For example, the structure may be a structure in social infrastructure such as a road (e.g., a road surface, a sign, and a ceiling and a side wall of a tunnel or the like), a railway, a harbor, a dam, and a communication facility. Alternatively, the structure may be a structure in a life-related social capital such as a school, a hospital, a park, and a social welfare facility.

In each example embodiment, the deterioration degree may be calculated using information other than the image. For example, each example embodiment may calculate the deterioration degree by using acceleration detected using an acceleration sensor or the like. In each example embodiment, the deterioration degree may be calculated not for each portion but for the entire structure.

The value range of the deterioration degree is optional.

For example, each example embodiment may use a crack rate of a road surface for expressing the deterioration degree. In this case, the value of deterioration degree falls within 0.0 to 1.0 (0% to 100%).

Alternatively, each example embodiment may use a rutting amount for expressing the deterioration degree. In this case, the value of the deterioration degree is generally an integer of 0 or more (the unit is mm). A rational number may be used as the value of the rutting amount.

Alternatively, in each example embodiment, International Roughness Index (IRI) may be used for expressing the deterioration degree. In this case, the value of the deterioration degree is generally a rational number of 0 or more (the unit is mm/m).

Alternatively, in each example embodiment, Maintenance Control Index (MCI) may be used for expressing the deterioration degree. The MCI is a composite deterioration index that can be obtained from a cracking rate, a rutting amount, and flatness.

As described, the value range of the deterioration degree is optional. The user of each example embodiment may appropriately select the deterioration in accordance with the deterioration degree of a structure to be repaired.

In the following description, a crack rate will be used as an example of expressing the deterioration degree. Therefore, in the following description, when the deterioration degree increases, the value thereof increases. However, as the value of the deterioration degree, a numerical value in which the value decreases commensurately when getting worse may be used in relation to processing using the deterioration degree.

### First Example Embodiment

Next, a first example embodiment of the present invention will be described with reference to the drawings.

### Description of Configuration

First, a configuration of a deterioration diagnosis device 100 according to the first example embodiment will be described with reference to a drawing.

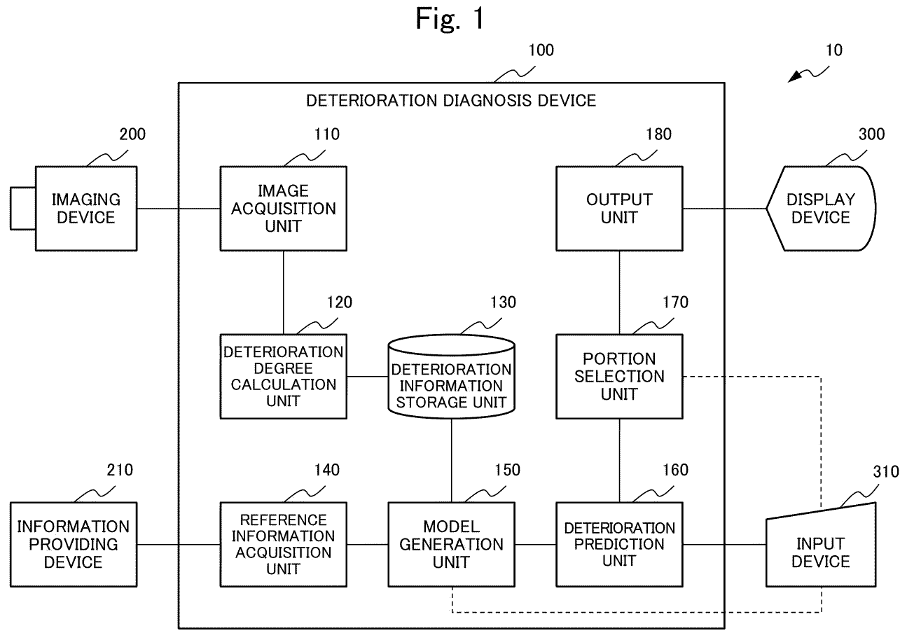

FIG. 1 is a block diagram illustrating an example of a configuration of a deterioration diagnosis system 10 including the deterioration diagnosis device 100 according to the first example embodiment.

The deterioration diagnosis system 10 includes the deterioration diagnosis device 100, an imaging device 200, an information providing device 210, a display device 300, and an input device 310.

The imaging device 200 captures an image including a portion to be diagnosed in a structure (e.g., a road surface, a sign, a ceiling, and/or a side wall).

The deterioration diagnosis system 10 can use any device as the imaging device 200 as long as the device can capture an image including a portion to be diagnosed. For example, the deterioration diagnosis system 10 may use a drive recorder installed for the purpose of recording the situation at the time of occurrence of an automobile accident as the imaging device 200. Alternatively, the deterioration diagnosis system 10 may use a camera (e.g., an omnidirectional camera) that captures a scene as the imaging device 200.

Alternatively, the imaging device 200 may be an imaging device mounted on a vehicle used in an intelligent transport system (ITS) or the like. The ITS is a transportation system using information technology (IT).

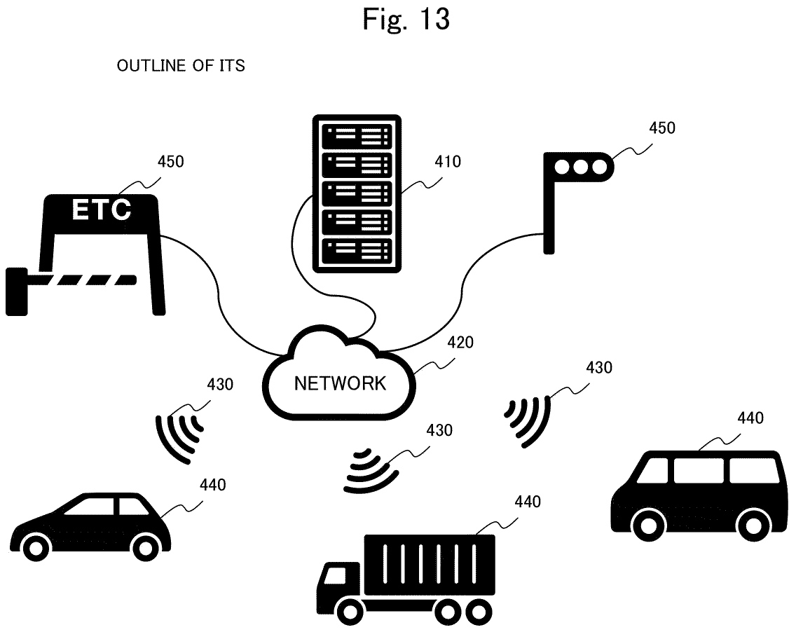

FIG. 13 is a diagram illustrating an outline of the ITS.

An information processing device 410 collects information from vehicles 440 via a network 420 and/or communication paths 430. Then, the information processing device 410 controls facilities 450 installed on a road or the like based on the collected information and executes predetermined processing (e.g., assistance of safe driving or management of roads). The facilities 450 are optional. FIG. 13 illustrates a traffic light and an electronic toll collection system (ETC in FIG. 13) as examples of the facilities 450.

Alternatively, the deterioration diagnosis system 10 may use a camera used for automatic driving as the imaging device 200. As described above, the deterioration diagnosis system 10 may be used in an automatic driving system.

The description returns to the description referring to FIG. 1.

Then, the imaging device 200 transmits the captured image to the deterioration diagnosis device 100 together with the capturing time.

The deterioration diagnosis device 100 may include the imaging device 200.

The information providing device 210 transmits the reference information used for generating the deterioration prediction model to the deterioration diagnosis device 100. The detail of the reference information will be described later.

The information providing device 210 may be a single device or a system including a plurality of devices. Alternatively, the information providing device 210 is not limited to a specific device, and may be achieved by using an information service enabled using computer resources connected via a predetermined network such as cloud computing.

The input device 310 receives, for the deterioration diagnosis device 100, an input of a date and time (hereinafter, also referred to as “prediction time”) for predicting deterioration from a user or the like. Then, the input device 310 transmits the received prediction time to the deterioration diagnosis device 100.

The input device 310 may receive, in addition to the prediction time, an input of information that is different from the prediction time and transmit it to the deterioration diagnosis device 100.

For example, the input device 310 may receive an input of information (hereinafter, may be called “selection condition”) related to selection of portions for the deterioration diagnosis device 100. Alternatively, the input device 310 may receive an input of information for the deterioration diagnosis device 100 to generate a deterioration prediction model. In any case, the input device 310 transmits the received information to the deterioration diagnosis device 100.

The input device 310 may display information necessary for receiving an input. For example, the input device 310 may include a display device such as a liquid crystal display. Alternatively, the input device 310 may cooperate with the display device 300 to receive an input.

The deterioration diagnosis device 100 may include the input device 310. For example, the input device 310 may be a keyboard, a mouse, or a touch pad.

The display device 300 receives an output (at least information related to a portion selected at a prediction time and deterioration degree of the portion) from the deterioration diagnosis device 100 to be described later, and displays the portion by using the received output from the deterioration diagnosis device 100.

The deterioration diagnosis system 10 can use any device as the display device 300 as long as the device can display the output from the deterioration diagnosis device 100. For example, the deterioration diagnosis system 10 may use, as the display device 300, a display device included in a system that manages repair and mending of a road. Alternatively, the deterioration diagnosis system 10 may use a display device of a terminal device (e.g., a liquid crystal display of a terminal) carried by a user as the display device 300.

The deterioration diagnosis device 100 may include the display device 300. For example, the display device 300 may be a liquid crystal display, an organic electroluminescence display, or electronic paper.

As described above, the display device 300 may display information that assists input for the input device 310.

Alternatively, the display device 300 and the input device 310 may be included in one device instead of different devices. For example, the display device 300 and the input device 310 may be achieved by using a computer device including a liquid crystal display, a keyboard, and a mouse. Alternatively, the display device 300 and the input device 310 may be achieved by using a touch panel including a touch pad and a liquid crystal display.

Furthermore, the display device 300, the input device 310, and the information providing device 210 may be included in one device.

The deterioration diagnosis device 100 acquires an image from the imaging device 200. Then, the deterioration diagnosis device 100 calculates the deterioration degree of the portion to be diagnosed included in the image. Then, the deterioration diagnosis device 100 stores the calculated deterioration degree as a history based on the capturing time.

Further, the deterioration diagnosis device 100 acquires reference information used for creating a deterioration prediction model from the information providing device 210. Then, the deterioration diagnosis device 100 generates a deterioration prediction model for predicting deterioration based on the history. Further, the deterioration diagnosis device 100 may generate the deterioration prediction model by using the reference information in addition to the history.

The deterioration diagnosis device 100 may use a statistic obtained from a statistically collected deterioration distribution as information for generating the deterioration prediction model. The deterioration diagnosis device 100 can generate a more reliable deterioration prediction model based on the statistically collected deterioration distribution. An acquisition source of the statistic is optional. For example, the deterioration diagnosis device 100 may acquire the statistic from a predetermined device. Alternatively, the deterioration diagnosis device 100 may calculate the statistic by applying predetermined processing to the calculated deterioration degree and/or history.

Further, the deterioration diagnosis device 100 receives the prediction time from the input device 310. Then, the deterioration diagnosis device 100 predicts the deterioration at the prediction time by using the deterioration prediction model. The deterioration diagnosis device 100 selects a portion relevant to deterioration meeting a predetermined condition (selection condition) among the predicted deterioration. Then, the deterioration diagnosis device 100 outputs information related to the selected portion (e.g., information related to the location of the selected portion) to the display device 300.

Next, a configuration of the deterioration diagnosis device 100 will be described.

The deterioration diagnosis device 100 includes an image acquisition unit 110, a deterioration degree calculation unit 120, a deterioration information storage unit 130, a reference information acquisition unit 140, a model generation unit 150, a deterioration prediction unit 160, a portion selection unit 170, and an output unit 180.

The image acquisition unit 110 acquires an image including a portion of a structure to be diagnosed (e.g., a road surface of a road, or a side wall and a ceiling of a tunnel) and a capturing time of the image. The image acquisition unit 110 may acquire information related to the location of the portion to be diagnosed (hereinafter, referred to as “location information”). The location information is, for example, the latitude and longitude of the portion. The location information may include a direction of the portion.

The deterioration degree calculation unit 120 calculates the deterioration degree of the portion to be diagnosed by using a predetermined method.

A method used by the deterioration degree calculation unit 120 to calculate the deterioration degree is optional. For example, the deterioration degree calculation unit 120 calculates the area of a road surface and the area of a crack included in the image by using predetermined image recognition. Then, the deterioration degree calculation unit 120 calculates a crack rate of the road surface as the deterioration degree based on the calculated area of the road surface and the calculated area of the crack.

The deterioration degree calculation unit 120 may calculate the deterioration degree by using predetermined machine learning or artificial intelligence.

The deterioration degree calculation unit 120 may determine the type of deterioration (e.g., cracking or rutting) included in the image by using predetermined image recognition, machine learning, or artificial intelligence, and calculate the deterioration degree in the determined deterioration. The image may include capturing time and location information as the information.

In some cases, the image may include a plurality of portions as diagnosis targets. In this case, the deterioration degree calculation unit 120 may calculate the deterioration degrees for all the portions. Alternatively, the deterioration degree calculation unit 120 may calculate the deterioration degree for a portion selected according to a predetermined selection rule.

The deterioration degree calculation unit 120 uses the calculated deterioration degree and the capturing time to store the history of the deterioration degree relevant to the portion in the deterioration information storage unit 130.

When there is a plurality of portions to be diagnosed, the deterioration degree calculation unit 120 stores a history relevant to each portion in the deterioration information storage unit 130. For example, the deterioration degree calculation unit 120 may store the history of the deterioration degree at each portion by using the location information of the portion to be diagnosed.

The acquisition source of the location information in the deterioration diagnosis device 100 is optional. For example, the image acquisition unit 110 may acquire the location information from the imaging device 200. Alternatively, a location calculation device (not illustrated) may calculate the location information by using the acquired image and map information in which the location and the image are associated with each other.

The deterioration information storage unit 130 stores a history of deterioration degree.

The reference information acquisition unit 140 acquires reference information related to deterioration of a portion from the information providing device 210. The reference information is used to generate a deterioration prediction model.

The reference information is optional. The reference information is determined in accordance with a target of deterioration prediction, types of deterioration to be predicted, a deterioration prediction model to be generated, and the like.

Examples of the reference information will be described.

For example, the traffic volume of the road affects the progress of deterioration. Therefore, the traffic volume in the portions to be subjected to the deterioration prediction serves as reference information for the deterioration prediction of the road.

Alternatively, vehicle weight affects deterioration of the road surface. Therefore, information related to vehicle weight (e.g., the ratio of large, medium, semi-medium, and standard vehicles) severs as reference information.

Alternatively, the weight of the load in the vehicle affects deterioration of the road surface. In general, a vehicle in commercial use tends to be loaded with heavier luggage than a vehicle in private use. Therefore, information related to the types of vehicles (e.g., the ratio of commercial to private vehicles) serves as reference information.

The types of vehicles can be determined with reference to characters, colors, and the like of the license plate. Therefore, the deterioration diagnosis device 100 may determine the characters and colors of the license plate included in the acquired image by using predetermined image recognition, machine learning, or artificial intelligence, and calculate the ratio of the vehicle types or the like. As described above, the deterioration diagnosis device 100 may generate a portion of the reference information. However, the determination of the vehicle type is not limited to the processing performed in the deterioration diagnosis device 100. For example, the imaging device 200 or a device (not illustrated) may generate the reference information based on the vehicle type.

Alternatively, the structure and material of the portion of to be diagnosed affect the progress of deterioration. Therefore, the structure and material of the portions to be diagnosed serve as reference information.

Alternatively, the type and timing of repairs performed in the past would affect the progress of deterioration in the future. Therefore, the type and timing of past repairs serve as reference information.

The model generation unit 150 generates a deterioration prediction model that predicts the deterioration degree of the portion at the designated time by using the history. The model generation unit 150 may generate a deterioration prediction model that predicts the deterioration degree of the portion at the designated time by using the reference information in addition to the history.

The model generation unit 150 may generate a deterioration prediction model used for the entire portions to be diagnosed. Alternatively, the model generation unit 150 may generate a deterioration prediction model for each portion to be diagnosed.

For example, the environment and other factors differ among the portions to be diagnosed. Alternatively, the structure may differ among the portions to be diagnosed.

For example, if the diagnosis target is a road, the type and number of vehicles traveling on each road will differ. In addition, the material of the road surface (e.g., concrete or asphalt) may be different for each road. Therefore, deterioration that is likely to occur may be different in each portion to be diagnosed.

Therefore, the model generation unit 150 may generate a deterioration prediction model for each portion. For example, even when a deterioration prediction model associated with the same deterioration is generated, the model generation unit 150 may generate a deterioration prediction model having different parameter values for each portion.

Furthermore, the model generation unit 150 may generate a deterioration prediction model in accordance with different deterioration for each portion.

The acquisition source of the information on the deterioration of each portion is optional. For example, the reference information acquisition unit 140 may acquire, as the reference information, information regarding a type of each portion or deterioration that is likely to occur in each portion. Alternatively, deterioration degree calculation unit 120 may determine the occurrence of deterioration based on an image used for calculating the deterioration degree. Alternatively, the model generation unit 150 may determine deterioration that is likely to occur based on the history.

Alternatively, the model generation unit 150 may divide portions be diagnosed into a plurality of groups based on a user’s instruction, a predetermined standard, or the like, and generate a deterioration prediction model for each group.

The model generation unit 150 may generate a deterioration prediction model that calculates further another piece of information in addition to the deterioration degree. For example, the model generation unit 150 may generate a deterioration prediction model that calculates the deterioration degree and deterioration speed.

Alternatively, the model generation unit 150 may generate, in addition to the deterioration degree, a deterioration prediction model that predicts a time at which predetermined deterioration occurs. The predetermined deterioration is optional. For example, the model generation unit 150 may generate a deterioration prediction model that predicts a time at which a linear crack, a tortoise-shell crack, and/or a pot hole will occur.

The model generation unit 150 may generate a deterioration prediction model associated with each type of deterioration. For example, the model generation unit 150 may generate a linear model for the occurrence of certain deterioration and generate a deterioration prediction model including a quadratic function for the occurrence of another type of deterioration. For example, the model generation unit 150 may generate a linear deterioration prediction model for the occurrence of a linear crack, and may generate a deterioration prediction model including a quadratic function for the occurrence of a tortoise-shell crack. Furthermore, the model generation unit 150 may generate a deterioration prediction model that integrates (e.g., synthesizes using weights) deterioration prediction models for multiple occurrences of deterioration.

The manner in which the model generation unit 150 generates the deterioration prediction model is optional. For example, the model generation unit 150 may store a reference model for generating a prediction model in advance, and generate a deterioration prediction model as a solution to an optimization problem including the reference model, a history, and reference information. Furthermore, the model generation unit 150 may use predetermined machine learning or artificial intelligence to generate the deterioration prediction model.

The model generation unit 150 may create a new deterioration prediction model instead of rewriting the deterioration prediction model. For example, when acquiring information of “repaired” as the reference information, the model generation unit 150 may generate a deterioration prediction model to be used after repair. The deterioration prediction model may include information related to cost regarding repair or the like. The user can grasp the cost effectiveness and the like by referring to the cost calculated using the deterioration prediction model.

The model generation unit 150 may receive information related to generation of the deterioration prediction model from the input device 310. For example, when the model generation unit 150 stores a plurality of reference models, the model generation unit 150 may receive designation of a reference model to be used for generation of a deterioration prediction model from the input device 310. Alternatively, the model generation unit 150 may receive the values and/or constraints of at least some parameters included in the deterioration prediction model from the input device 310.

When the deterioration diagnosis device 100 handles a plurality of types of deterioration (for example, a cracking rate and a rutting amount), the model generation unit 150 may generate a deterioration prediction model for each deterioration type.

Alternatively, the model generation unit 150 may generate a deterioration prediction model that predicts a deterioration degree obtained by integrating a plurality of deteriorations. For example, the model generation unit 150 may generate a deterioration prediction model that calculates a deterioration degree obtained by integrating each deterioration degree by using a predetermined weight associated with each deterioration.

The weight for each deterioration may be fixed. Alternatively, the model generation unit 150 may set a weight for each deterioration in generation of the deterioration prediction model.

The deterioration prediction unit 160 receives the prediction time from the input device 310. Then, the deterioration prediction unit 160 calculates the deterioration degree at the prediction time by using the deterioration prediction model.

In a case when there are a plurality of portions to be predicted, the deterioration prediction unit 160 may receive designation of a portion for predicting the deterioration degree from the input device 310. The designation of the portion may be designation including a plurality of portions. For example, the deterioration prediction unit 160 may receive designation of portions included in a predetermined range (for example, one or a plurality of management units in a road) and predict the deterioration degrees of the portions included in the range.

When the model generation unit 150 generates a plurality of deterioration prediction models, the deterioration prediction unit 160 predicts the deterioration degree by using all the deterioration prediction models. However, the deterioration prediction unit 160 may use some of the deterioration prediction models. For example, the deterioration prediction unit 160 may receive designation of a deterioration prediction model used for prediction from the input device 310.

The portion selection unit 170 selects a portion where the predicted deterioration degree meets the selection condition from among portions for which the deterioration degree has been predicted.

The portion selection unit 170 may receive the selection condition from the input device 310. Alternatively, the portion selection unit 170 may use a selection condition set in advance by a user or the like.

The selection condition may include a plurality of conditions.

For example, the portion selection unit 170 may use the extent of the deterioration degree (for example, the deterioration degree is LARGE) and the range of the portion (for example, designation of a road) as the selection condition.

Alternatively, for example, when the deterioration prediction model calculates the deterioration degree and the deterioration speed, the portion selection unit 170 may use the extent of the deterioration degree (for example, the deterioration degree is LARGE) and the magnitude of the deterioration speed (for example, the deterioration speed is HIGH) as the selection condition.

Alternatively, when the deterioration prediction model calculates, in addition to the deterioration degree, the timing at which predetermined deterioration (for example, a pot hole) will occur, the portion selection unit 170 may use the occurrence of the predetermined deterioration in addition to the extent of deterioration degree as the selection condition.

The portion selection unit 170 may use a selection condition including three or more conditions.

Then, the portion selection unit 170 outputs the selected portion to the output unit 180.

The output unit 180 outputs information related to the selected portion and the deterioration degree of the portion at the prediction time. The output unit 180 may output information related to the unselected portion instead of the information related to the selected portion. In the following description, as an example, the output unit 180 outputs “information related to the selected portion”.

The content of the information output by the output unit 180 is optional. The user of the deterioration diagnosis device 100 may select information to be output according to the output destination.

An example of information output by the output unit 180 will be described.

For example, in a case when the display device 300 displays the deterioration degree at the prediction time for the selected portion on a map, the output unit 180 may output the location information (for example, latitude and longitude) and the deterioration degree of the selected portion.

In outputting the information related to the selected portion, the output unit 180 may appropriately acquire the information from the configuration in which the information is stored or the configuration in which the information can be output. For example, when the location information is output, the output unit 180 may acquire the location information from the image acquisition unit 110 or the deterioration information storage unit 130.

### Description of Operations

First, an operation of the deterioration diagnosis device 100 according to the first example embodiment will be described with reference to a drawing.

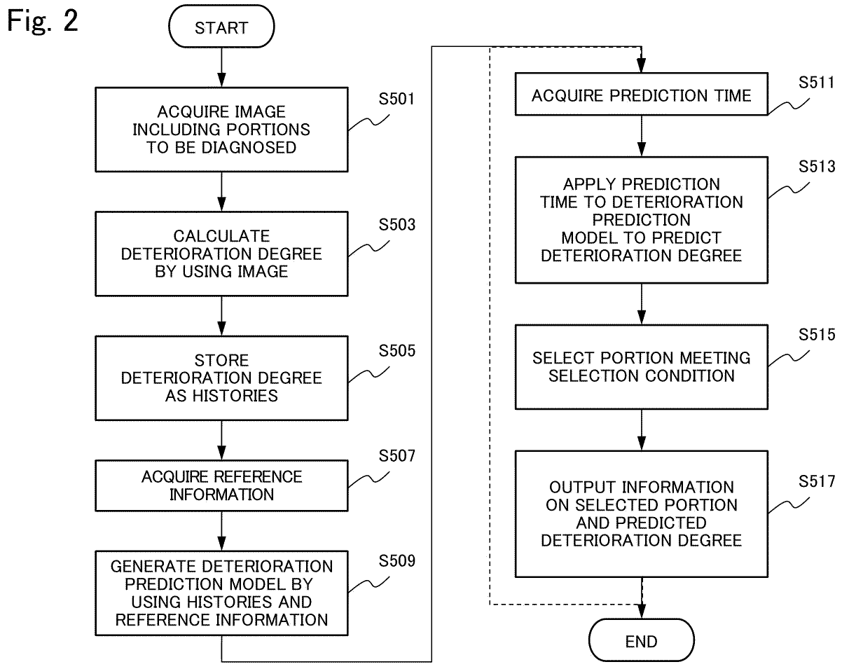

FIG. 2 is a flowchart illustrating an example of operation of the deterioration diagnosis device 100.

The image acquisition unit 110 acquires an image including portion to be diagnosed (step S501).

The deterioration degree calculation unit 120 calculates deterioration degree by using the image (step S503).

The deterioration information storage unit 130 stores deterioration degree as histories (step S505).

The reference information acquisition unit 140 acquires reference information (step S507).

The model generation unit 150 generates a deterioration prediction model by using the histories and the reference information (step S509).

The deterioration prediction unit 160 acquires a prediction time (step S511).

The deterioration prediction unit 160 applies the prediction time to the deterioration prediction model to predict the deterioration degree (step S513).

The portion selection unit 170 selects a portion meeting the selection condition (step S515).

The output unit 180 outputs the information on the selected portion and the predicted deterioration degree (step S517).

Then, the deterioration diagnosis device 100 ends the operation.

The deterioration diagnosis device 100 may repeat the operations of steps S511 to S517.

For example, after operating to Step S509, the deterioration diagnosis device 100 waits for reception of the prediction time from the input device 310. When receiving the prediction time from the input device 310, the deterioration diagnosis device 100 operates the steps from steps S511 to S517. Then, the deterioration diagnosis device 100 may again wait for reception of the prediction time from the input device 310.

### Specific Examples

Next, the operation of the deterioration diagnosis device 100 will be described with reference to specific examples.

In the following description, it is assumed that the display device 300 and the input device 310 may be achieved by using a computer device including a liquid crystal display, a keyboard, and a mouse.

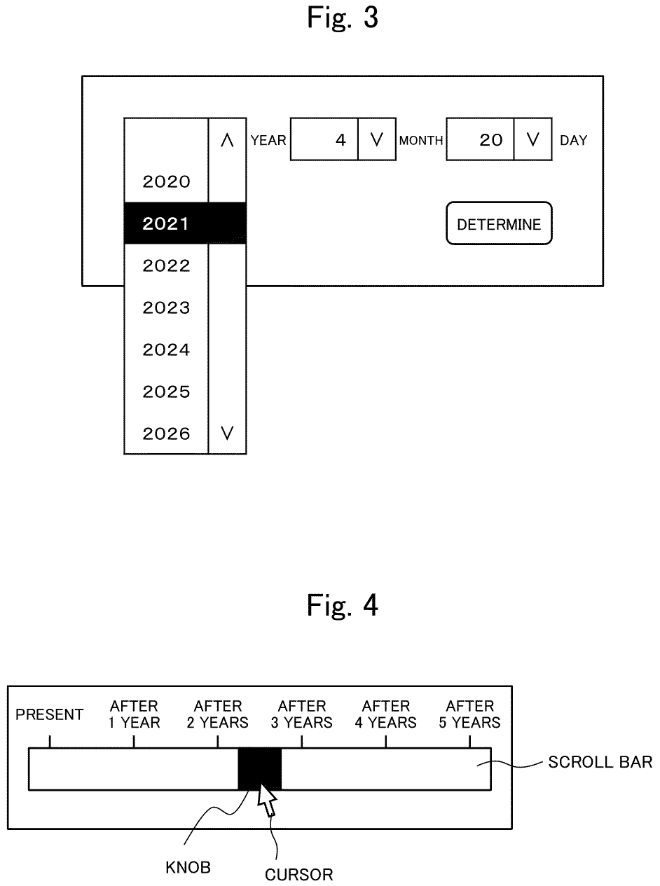

FIG. 3 is a diagram illustrating an example of display for inputting a prediction time. FIG. 3 uses a pull-down menu as an input of the prediction time. The user uses the pull-down menu to set the year, month, and day of the prediction time. Then, the user operates the mouse or the like to place the cursor on the “determine” button, and presses (clicks) the button such as the mouse. When detecting the pressing of the button, the input device 310 transmits the displayed prediction time to the deterioration diagnosis device 100.

The input of the prediction time is not limited to the pull-down menu. For example, the display device 300 displays a numerical value input form. Then, the input device 310 may receive a numerical value input to the form based on an operation of a keyboard or the like.

Alternatively, the display device 300 may display a scroll bar indicating the prediction time. In this case, the input device 310 refers to the position of the knob on the scroll bar and receives the prediction time.

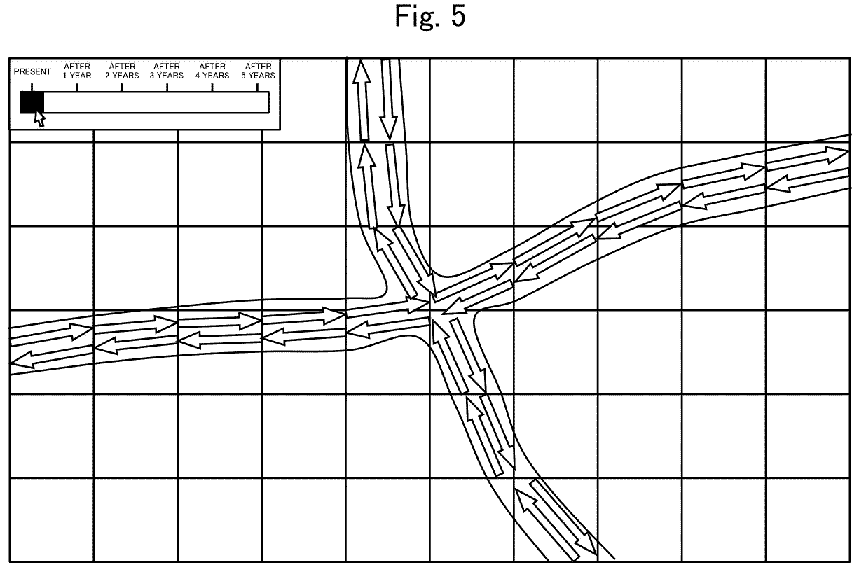

FIG. 4 is a diagram illustrating another example of display for inputting a prediction time. FIG. 4 is an example of display in a case when a scroll bar is used.

The input of the prediction time using the display illustrated in FIG. 4 will be described.

The user operates the mouse to place the cursor on the knob, and presses a button on the mouse or the like in the overlapped state. Then, the user moves the cursor in one of the left and right directions while pressing the button, and moves the knob to a position of a desired prediction time.

Then, the input device 310 transmits the prediction time relevant to the knob position to the deterioration diagnosis device 100.

The input device 310 may continuously transmit the prediction time. Alternatively, the input device 310 may transmit the prediction time at a predetermined cycle. Alternatively, the input device 310 may transmit the prediction time at the timing when the user releases the button. Alternatively, the input device 310 may transmit the prediction time in a case when the prediction time changes (specifically, in a case when the position of the knob has been changed).

When receiving the prediction time, the deterioration diagnosis device 100 outputs the information on the portion selected at the prediction time and the deterioration degree of the portion at the prediction time.

Next, the output from the deterioration diagnosis device 100 will be described with reference to the drawings.

In the following description, a diagnosis target is a road. The selection condition is “deterioration degree is LARGE”. Furthermore, the present time is assumed as being after repair.

The deterioration diagnosis device 100 outputs information related to a portion meeting the selection condition. Therefore, the display device 300 may display the deterioration degree of the portion meeting the selection condition. However, the display on the display device 300 is not limited to the above. For example, the display device 300 may change the display of the portion meeting the selection condition.

Further, the deterioration diagnosis system 10 may use a plurality of selection conditions. In this case, the display device 300 may use a display relevant to a plurality of selection conditions.

The diagram used in the following description also displays a portion that does not satisfy the selection condition (in this case, “deterioration degree is LARGE”) in order to facilitate comparison of outputs in different prediction times.

In each drawing, the color of the arrow indicates the predicted deterioration degree. Black arrows indicate the portions each diagnosed that the deterioration degree is LARGE. Gray arrows indicate the portions each diagnosed that the deterioration degree is MEDIUM. White arrows indicate the portions each diagnosed that the deterioration degree is SMALL. In other words, the black arrows indicate the selected portions. The other arrows indicate portions that have not been selected.

That is, in each drawing, the colors of the arrows indicate the deterioration degree. The black arrows indicate the selected portions.

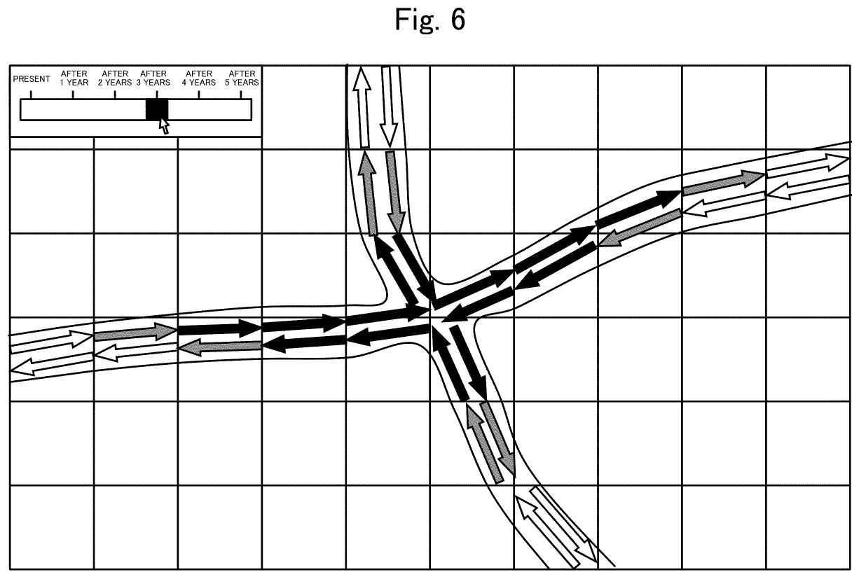

FIG. 5 is a diagram illustrating a first example of display of portions. FIG. 5 is a diagram illustrating a present state. The present time is assumed as being after repair. Therefore, in FIG. 5, all the portions are in a state where the deterioration degree is low state.

The scroll bar in the upper left portion of FIG. 5 is a display to be used for inputting in the input device 310 described with reference to FIG. 4.

FIG. 6 is a diagram illustrating a second example of display of the portions. FIG. 6 illustrates a prediction of the deterioration degree after three years.

In FIG. 6, black arrows indicate the portions each diagnosed that the deterioration degree is LARGE, e.g., the selected portions. Gray arrows indicate the portions each diagnosed that the deterioration degree is MEDIUM. White arrows indicate the portions each diagnosed that the deterioration degree is SMALL. That is, the gray arrows and the white arrows indicate the portions that have not been selected.

For example, when the user moves the prediction time from the present to 3 years later by using the slide tab, the input device 310 transmits the prediction time (for example, the date after three years) to the deterioration diagnosis device 100. The deterioration diagnosis device 100 outputs the deterioration degree of each portion at the prediction time. The display device 300 displays the deterioration degree of each portion at the prediction time.

As described above, the display device 300 changes the display related to the portions by using the information related to the portions and the deterioration degree output from the deterioration diagnosis device 100.

The user can grasp the change in the deterioration degree by using the deterioration diagnosis system 10.

The display device 300 may display deterioration values (for example, the cracking rate, the rutting amount, or IRI) of the portions to be diagnosed.

Furthermore, in a case when the deterioration prediction model predicts occurrence of predetermined deterioration, the deterioration diagnosis system 10 may display occurrence of predetermined deterioration.

For example, when the user moves the slide tab of the prediction time, the deterioration diagnosis device 100 outputs the predetermined deterioration occurring at the prediction time associated with the movement and the information related to the relevant portion. The display device 300 displays, on the portions where the predetermined deterioration occurs, the occurring predetermined deterioration by using the output from the deterioration diagnosis device 100.

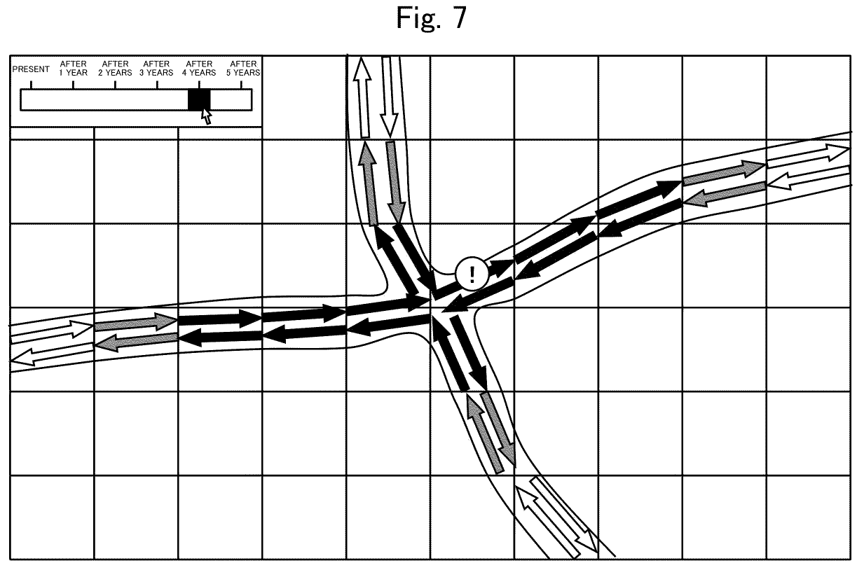

FIG. 7 is a diagram illustrating display examples of the predetermined deterioration. The predetermined deterioration is, for example, a linear crack, a tortoise-like crack, or a pot hole. FIG. 7 illustrates a portion where predetermined deterioration has occurred, indicated by symbol “!”.

The user of the deterioration diagnosis system 10 can refer to the deterioration while changing the prediction time. Therefore, the user can grasp the prediction time of occurrence of predetermined deterioration by using the deterioration diagnosis system 10.

FIGS. 5 to 7 also illustrate the portions that have not been selected. The display device 300 does not necessarily display the unselected portion.

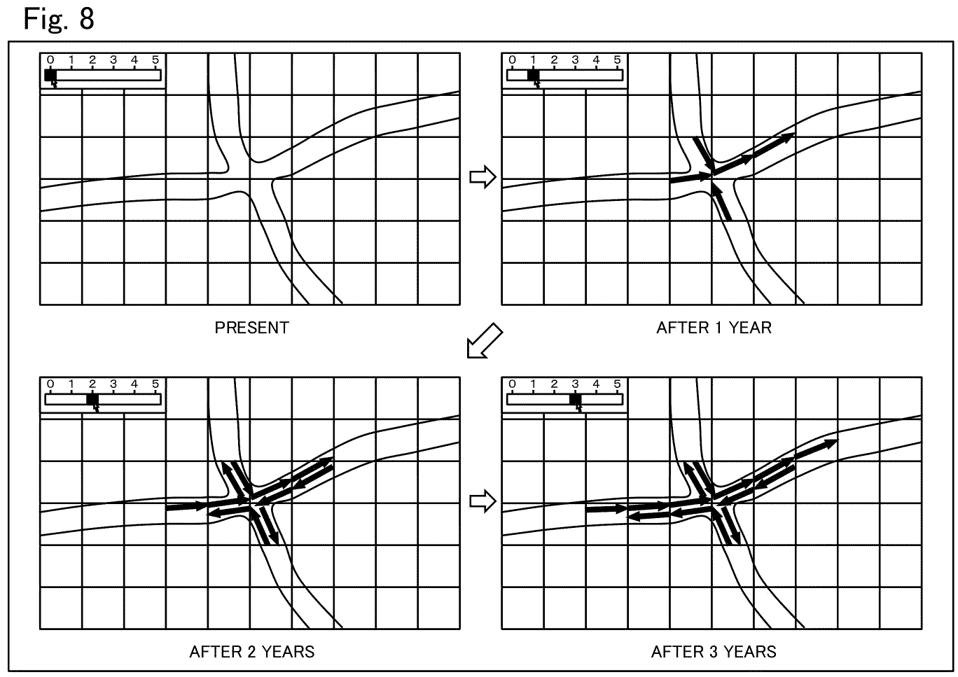

FIG. 8 is a diagram illustrating an example of displaying the selected portions. FIG. 8 illustrates changes in display associated with the portions diagnosed that the deterioration degree is LARGE for the period from the present to after three years.

The user can grasp the occurrence and progress of the deterioration with reference to the display as illustrated in FIG. 8.

The deterioration diagnosis system 10 may collectively execute the processing as illustrated in FIG. 8. For example, the deterioration diagnosis system 10 may execute the following operations.

The input device 310 transmits a plurality of prediction times to the deterioration diagnosis device 100. The deterioration diagnosis device 100 outputs information related to the portion selected in each of the received prediction times and the deterioration degree of the portion at the prediction time to the display device 300. Then, the display device 300 changes the display of the information related to the portions and the deterioration degree output from the deterioration diagnosis device 100 along the prediction time in response to the instruction of the user or the like.

Further, the display device 300 may continuously change the display. For example, the display device 300 may continuously display the deterioration degree associated with a plurality of times as a moving image.

### Description of Effects

Next, the effects of the deterioration diagnosis device 100 according to the first example embodiment will be described.

The deterioration diagnosis device 100 according to the first example embodiment can exert the effects of selecting a portion meeting a predetermined condition at the prediction time from among portions for which deterioration has been predicted.

The reason is as follows.

The deterioration diagnosis device 100 includes the deterioration information storage unit 130, the model generation unit 150, the deterioration prediction unit 160, the portion selection unit 170, and the output unit 180. The deterioration information storage unit 130 stores deterioration degree histories for portions of a structure to be diagnosed. The model generation unit 150 generates, based on the histories, a deterioration prediction model for predicting the deterioration degree of the portions. The deterioration prediction unit 160 predicts the deterioration degree of the portions at a prediction time by using the generated deterioration prediction model. The portion selection unit 170 selects, from among the portions, a portion where the deterioration degree predicted at a prediction time meets a predetermined condition. The output unit 180 outputs information relating to the selected portion and the predicted deterioration degree of the selected portion.

That is, the deterioration diagnosis device 100 generates, based on the deterioration degree histories for the portions to be diagnosed, the deterioration prediction model that predicts the deterioration of the portion. Then, the deterioration diagnosis device 100 predicts the deterioration degree at the prediction time by using the deterioration prediction model. Then, the deterioration diagnosis device 100 selects the portion where the predicted deterioration degree meets a predetermined condition, and outputs information related to the selected portion and the deterioration degree.

The user of the deterioration diagnosis device 100 can grasp a portion meeting a predetermined condition at the prediction time (for example, a portion appropriate as a target of repair or the like, such as a portion diagnosed that the deterioration degree is LARGE) from among the portions for which the deterioration has been predicted.

In addition, the deterioration diagnosis device 100 may generate, based on the history of deterioration degree of each portion to be diagnosed, a deterioration prediction model that predicts deterioration of each portion. In this case, since the deterioration diagnosis device 100 uses the deterioration prediction model based on the deterioration history in each portion and the reference information related to the deterioration of the portion, it is possible to predict an appropriate deterioration degree of each portion.

Further, the deterioration diagnosis device 100 may generate the deterioration prediction model by using the reference information related to the deterioration of the portion. Therefore, the deterioration diagnosis device 100 can generate a more accurate deterioration prediction model. The deterioration diagnosis device 100 further includes the image acquisition unit 110 and the deterioration degree calculation unit 120. The image acquisition unit 110 acquires an image including a portion to be diagnosed. The deterioration degree calculation unit 120 calculates the deterioration degree relevant to the portion by using the image, and stores the calculated deterioration degree as the history in the deterioration information storage unit 130.

The deterioration diagnosis device 100 can store, by using these configurations, the history of the deterioration degree used to calculate the deterioration speed by using the image including the portion to be diagnosed.

The deterioration diagnosis system 10 includes the deterioration diagnosis device 100, the information providing device 210, the display device 300, and the input device 310. The information providing device 210 provides reference information to the deterioration diagnosis device 100. The input device 310 transmits a prediction time to the deterioration diagnosis device 100. The deterioration diagnosis device 100 outputs information related to the portion where the deterioration degree at the prediction time meets a predetermined condition (selection condition) and the deterioration degree based on the above-explained operations. The display device 300 displays the deterioration degree of the portion meeting the selection condition at the prediction time by using the information related to the portion and the deterioration degree output from the deterioration diagnosis device 100.

Based on such a configuration, the deterioration diagnosis system 10 can provide the user with information that enables selection of a portion meeting a predetermined condition at the prediction time.

The deterioration diagnosis system 10 further includes the imaging device 200. The imaging device 200 captures an image including a portion of a structure to be diagnosed, and transmits the image to the deterioration diagnosis device 100.

Based on such a configuration, the deterioration diagnosis system 10 can diagnose deterioration of a portion of a structure included in an image by using the image captured by the imaging device 200.

Note that, in the present example embodiment, an example has been described in which the deterioration degree calculation unit 120 calculates the deterioration degree by using the image acquired from the imaging device 200. However, instead of the imaging device 200, the deterioration degree calculation unit 120 may calculate the deterioration degree by using information acquired from an acceleration sensor (not illustrated). For example, the deterioration degree calculation unit 120 may calculate IRI as the deterioration degree in accordance with a change in acceleration acquired from the acceleration sensor. In this case, the model generation unit 150 may generate the deterioration prediction model by using the IRI stored as the deterioration degree.

Further, the deterioration degree calculation unit 120 may calculate the deterioration degree by using both the image acquired from the imaging device 200 and the information acquired from the acceleration sensor. In this case, the model generation unit 150 may generate the deterioration prediction model by using the cracking rate of the road surface and the IRI.

### Hardware Configuration

Next, a hardware configuration of the deterioration diagnosis device 100 will be described.

Each component of the deterioration diagnosis device 100 may be configured of a hardware circuit.

Alternatively, in the deterioration diagnosis device 100, each component may be configured by using a plurality of devices connected via a network.

Alternatively, in the deterioration diagnosis device 100, the plurality of components may be configured of one piece of hardware.

Alternatively, the deterioration diagnosis device 100 may be achieved as a computer device including a central processing unit (CPU), a read only memory (ROM), and a random access memory (RAM). In addition to the above configuration, the deterioration diagnosis device 100 may be achieved as a computer device including a network interface circuit (NIC). Furthermore, the deterioration diagnosis device 100 may be achieved as a computer device including a graphics processing unit (GPU) in order to speed up the deterioration diagnosis processing.

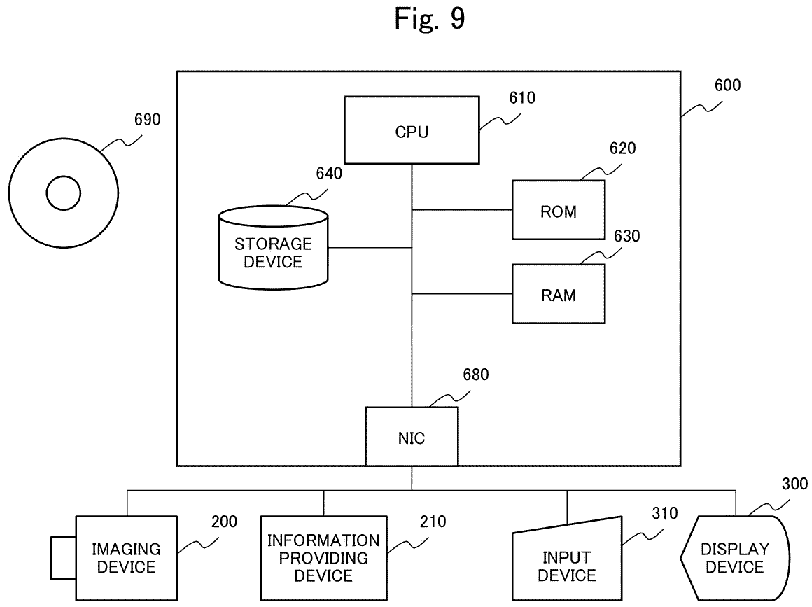

FIG. 9 is a block diagram illustrating a configuration of an information processing device 600 that is an example of a hardware configuration of the deterioration diagnosis device 100.

The information processing device 600 includes a CPU 610, a ROM 620, a RAM 630, a storage device 640, and an NIC 680, and constitutes a computer device.

The CPU 610 reads a program from the ROM 620 and/or the storage device 640. Then, the CPU 610 controls the RAM 630, the storage device 640, and the NIC 680 based on the read program. Then, the computer device including the CPU 610 controls these configurations and achieves the functions as each configuration illustrated in FIG. 1. Each configuration illustrated in FIG. 1 includes the image acquisition unit 110, the deterioration degree calculation unit 120, the deterioration information storage unit 130, the reference information acquisition unit 140, the model generation unit 150, the deterioration prediction unit 160, the portion selection unit 170, and the output unit 180.

For achieving each function, the CPU 610 may use the RAM 630 or the storage device 640 as a temporary storage medium of the program.

In addition, the CPU 610 may read the program included in the storage medium 690 storing the program so as to be readable by the computer device, by using a storage medium reading device (not illustrated). Alternatively, the CPU 610 may receive a program from an external device (not illustrated) via the NIC 680, store the program in the RAM 630 or the storage device 640, and operate based on the stored program.

The ROM 620 stores programs executed by the CPU 610 and fixed data. The ROM 620 is, for example, a programmable ROM (P-ROM) or a flash ROM.

The RAM 630 temporarily stores programs and data executed by the CPU 610. The RAM 630 is, for example, a dynamic-RAM (D-RAM).

The storage device 640 stores data and programs to be stored for a long period of time by the information processing device 600. The storage device 640 operates as the deterioration information storage unit 130. Furthermore, the storage device 640 may operate as a temporary storage device of the CPU 610. The storage device 640 is, for example, a hard disk device, a magneto-optical disk device, a solid-state drive (SSD), or a disk array device.

The ROM 620 and the storage device 640 are non-volatile (non-transitory) storage media. On the other hand, the RAM 630 is a volatile storage (transitory) medium. The CPU 610 is operable based on a program stored in the ROM 620, the storage device 640, or the RAM 630. That is, the CPU 610 can operate using a non-volatile storage medium or a volatile storage medium.

The NIC 680 mediates transmission and reception of data between the information processing device 600 and the imaging device 200, between the information processing device 600 and the information providing device 210, between the information processing device 600 and the display device 300, and between the information processing device 600 and the input device 310. The NIC 680 is, for example, a local area network (LAN) card. Furthermore, the NIC 680 is not limited to wired communication, and wireless communication may be used.

The information processing device 600 configured as described above can obtain the effects similar to those provided by the deterioration diagnosis device 100.

The reason is that the CPU 610 of the information processing device 600 can achieve a function similar to that of the deterioration diagnosis device 100 based on the program.

### Second Example Embodiment

As a second example embodiment, an outline of the deterioration diagnosis device 100 and the deterioration diagnosis system 10 according to the first example embodiment will be described.

### Description of Configuration

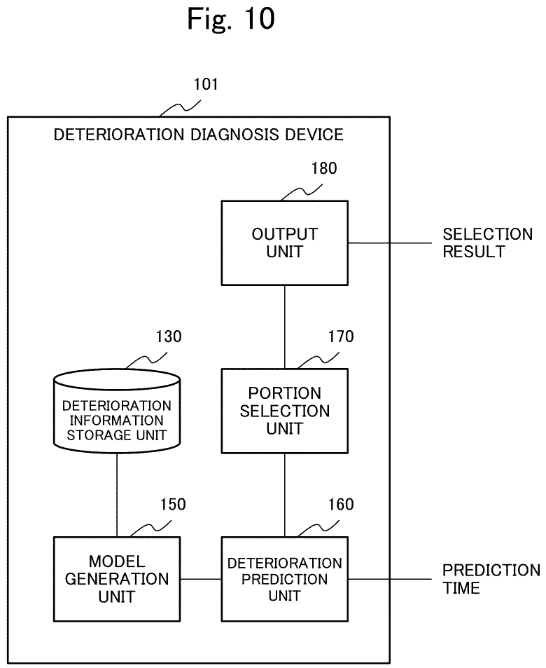

FIG. 10 is a block diagram illustrating an example of a configuration of a deterioration diagnosis device 101 according to a second example embodiment that is an outline of the deterioration diagnosis device 100 according to the first example embodiment.

The deterioration diagnosis device 101 includes a deterioration information storage unit 130, a model generation unit 150, a deterioration prediction unit 160, a portion selection unit 170, and an output unit 180. The deterioration information storage unit 130 stores deterioration degree histories for portions of a structure to be diagnosed. The model generation unit 150 generates, based on the histories, a deterioration prediction model for predicting the deterioration degree of the portions. The deterioration prediction unit 160 predicts the deterioration degree of the portions at a prediction time by using the generated deterioration prediction model. The portion selection unit 170 selects, from among the portions, a portion where the deterioration degree predicted at the prediction time meets a predetermined condition. The output unit 180 outputs information relating to the selected portion and the predicted deterioration degree of the selected portion.

The deterioration diagnosis device 101 may be achieved by using a computer device illustrated in FIG. 9.

### Description of Operations

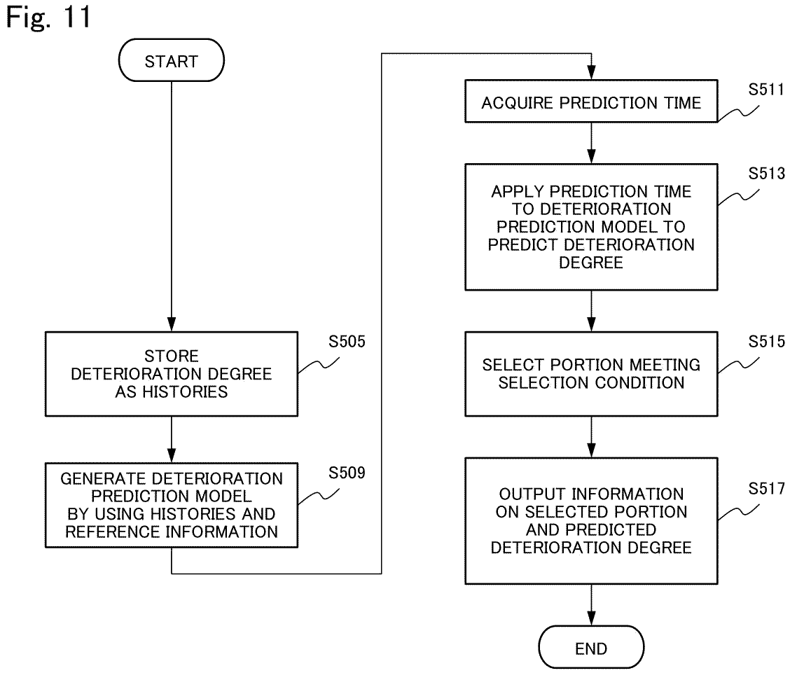

FIG. 11 is a flowchart illustrating an example of operation of the deterioration diagnosis device 101 according to the second example embodiment.

The deterioration information storage unit 130 stores deterioration degree as histories (step S505).

The model generation unit 150 generates a deterioration prediction model by using the histories (step S509).

The deterioration prediction unit 160 acquires a prediction time (step S511).

The deterioration prediction unit 160 applies the prediction time to the deterioration prediction model to predict the deterioration degree (step S513).

The portion selection unit 170 selects a portion meeting the selection condition (step S515).

The output unit 180 outputs the information on the selected portion and the deterioration degree (step S517).

Then, the deterioration diagnosis device 101 ends the operation.

As above described, the deterioration diagnosis device 101 generates, based on the deterioration degree histories for the portions to be diagnosed, the deterioration prediction model that predicts the deterioration of the portions. Then the deterioration diagnosis device 101 predicts the deterioration degree at the prediction time by using the deterioration prediction model. The deterioration diagnosis device 101 then selects the portion where the predicted deterioration degree meets a predetermined condition, and outputs information related to the selected portion and the deterioration degree.

The user of such a deterioration diagnosis device 101 can grasp a portion to be preferentially repaired, that is, a portion more appropriate as a target of repair or the like, among from the portions for which the deterioration has been predicted by using the deterioration degree at the prediction time.

### Description of Effects

Similar to the first example embodiment, the deterioration diagnosis device 101 can exert the effect of selecting a portion meeting a predetermined condition at the prediction time from among portions for which deterioration has been predicted.

This is because each configuration of the deterioration diagnosis device 101 operates similarly to the relevant configuration of the deterioration diagnosis device 100.

The deterioration diagnosis device 101 in FIG. 10 is the minimum configuration of the deterioration diagnosis device 100 according to the first example embodiment.

### Description of System

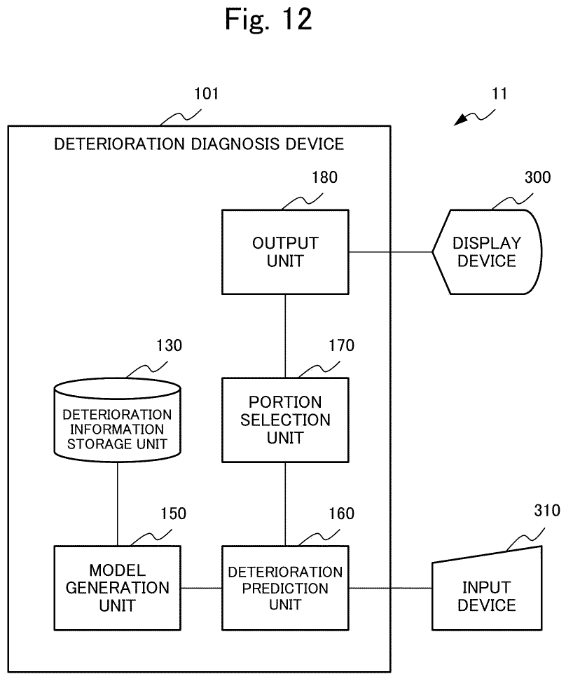

FIG. 12 is a block diagram illustrating an example of a configuration of a deterioration diagnosis system 11 including the deterioration diagnosis device 101 according to the second example embodiment.

The deterioration diagnosis system 11 includes a deterioration diagnosis device 101, a display device 300, and an input device 310. The input device 310 transmits a prediction time to the deterioration diagnosis device 101. The deterioration diagnosis device 101 outputs information related to the portion selected at the prediction time and the deterioration degree. The display device 300 displays the deterioration degree of the portion meeting the selection condition at the prediction time by using the information related to the portion and the deterioration degree output from the deterioration diagnosis device 101.

Based on such a configuration, the deterioration diagnosis system 11 can present a portion meeting a predetermined condition at the prediction time from among the portions to be subjected to the deterioration diagnosis to the user.

The deterioration diagnosis system 11 in FIG. 12 is the minimum configuration of the deterioration diagnosis system 10 according to the first example embodiment.

Although the present invention is described with reference to the example embodiments, the present invention is not limited to the above example embodiments. It will be understood by those of ordinary skill in the art that various changes in form and details may be made therein without departing from the sprit and scope of the invention as defined by the claims.

### Industrial Applicability

The present invention can be applied to a traffic system using information technology (IT) such as an intelligent transport system (ITS).

This application is based upon and claims the benefit of priority from Japanese patent application No. 2020-062913, filed on Mar. 31, 2020, the disclosure of which is incorporated herein in its entirety by reference.

### Reference Signs List

10 deterioration diagnosis system
11 deterioration diagnosis system
100 deterioration diagnosis device
110 image acquisition unit
120 deterioration degree calculation unit
130 deterioration information storage unit
140 reference information acquisition unit
150 model generation unit
160 deterioration prediction unit
170 portion selection unit
180 output unit
200 imaging device
210 information providing device
300 display device
310 input device
410 information processing device
420 network
430 communication path
440 vehicle
450 facility
600 information processing device
610 CPU
620 ROM
630 RAM
640 storage device
660 input apparatus
670 display apparatus
680 NIC
690 storage medium

What is claimed is:

1. A deterioration diagnosis device comprising:
a memory; and
at least one processor coupled to the memory,
the processor performing operations, the operations comprising:
storing deterioration degree histories for portions of a structure to be diagnosed;
generating, based on the histories, a deterioration prediction model for predicting the deterioration degrees of the portions;
predicting the deterioration degrees of the portions at a prediction time by using the generated deterioration prediction model;
selecting, from among the portions, a portion where the deterioration degree predicted at the prediction time meets a predetermined condition; and
outputting information relating to the selected portion and the predicted deterioration degree of the selected portion.

2. The deterioration diagnosis device according to claim 1, wherein the operations further comprise:
acquiring reference information related to deteriorations of the portions; and
generating the deterioration prediction model for predicting the deterioration degrees of the portions based on the histories and the reference information.

3. The deterioration diagnosis device according to claim 1, wherein the operations further comprise:
outputting, as information related to the selected portion, information related to a location of the portion.

4. The deterioration diagnosis device according to claim 1, wherein the operations further comprise:
generating the deterioration prediction model that predicts deterioration for each of the portions.

5. The deterioration diagnosis device according to claim 1, wherein the operations further comprise:
generating the deterioration prediction model that predicts a time at which predetermined deterioration occurs.

6. The deterioration diagnosis device according to claim 1, wherein the operations further comprise:
acquiring an image including the portion to be diagnosed;
calculating the deterioration degree relevant to the portion by using the image; and
storing the calculated deterioration degrees as the history.

7-8. (canceled)

9. A deterioration diagnosis method comprising:
storing deterioration degree histories for portions of a structure to be diagnosed;
generating, based on the histories, a deterioration prediction model for predicting the deterioration degrees of the portions;
predicting the deterioration degrees of the portions at a prediction time by using the generated deterioration prediction model;
selecting, from among the portions, the portion where the deterioration degree predicted at a prediction time meets a predetermined condition; and
outputting information related to the selected portion and the deterioration degree predicted at the selected portion.

10. A non-transitory computer-readable recording medium embodying a program, the program causing a computer to perform a method, the method comprising:
storing deterioration degree histories for portions in a structure to be diagnosed;
generating, based on the histories, a deterioration prediction model for predicting the deterioration degrees of the portions;
predicting the deterioration degrees of the portions at a prediction time by using the generated deterioration prediction model;
selecting, from among the portions, the portion where the deterioration degree predicted at the prediction time meets a predetermined condition; and
outputting information related to the selected portion and the deterioration degree predicted for the selected portion.
